(19) United States
(12) Patent Application Publication
(10) Pub. No.: US 2017/0224276 A1
(43) Pub. Date: Aug. 10, 2017

(54) Human Body Frequency Diagnostic Analysis Apparatus and Improvement Method of Health Assessment

(71) Applicant: TSE KANG BIOTECHNOLOGY CO., LTD, Kaohsiung City (TW)

(72) Inventor: Ming Chih He, Kaohsiung City (TW)

(21) Appl. No.: 15/015,172

(22) Filed: Feb. 4, 2016

(51) Int. Cl. A61B 5/00; A61B 5/024; A61B 5/0205

(52) U.S. Cl. CPC A61B 5/4854; A61B 5/0205; A61B 5/024

(57) ABSTRACT

Disclosed is a human body frequency diagnostic analysis apparatus including a transceiver with an end connected to a human head and driven to transmit a transmission frequency and drive the human head to feed back a feedback frequency, a detection unit connected to the transceiver for driving the transceiver to transmit the transmission frequency and receive the feedback frequency, and a control unit connected to the detection unit for controlling the detection unit and includes a computing unit for computing and comparing the transmission frequency with the feedback frequency to generate a dual line spectrum, a database for comparing the dual line spectrum to generate detected information, and a screen for displaying the dual line spectrum and the detected information. A maximum drop value detected in a frequency section of the dual line spectrum indicates an imbalance of a human body frequency.

### BACKGROUND OF THE INVENTION

Field of the Invention

The present invention relates to a human body frequency diagnostic analysis apparatus and an improvement method of health assessment, and more particularly to the human body frequency diagnostic analysis apparatus and improvement method of health assessment capable of diagnosing and analyzing a human body frequency and examining health conditions.

Description of the Related Art

In a theory of Chinese medicine, human body has a delicate distribution system responsible for transmitting microscopic matters such as “qi” and “blood”, and such transmission and distribution network is called a human meridian system for communicating internal organs and limbs and circulating qi and blood. The human meridian system has larger channels called “meridian channels” and smaller channels called “collateral channels”. Further, the theory of Chinese medicine also believes that each human organ has a frequency, and a frequency imbalance of an organ indicates a problem of the organ. Western medicine also advocates that prevention is better than cure, and suggests us to have regular medical check-ups. The main purpose of the regular medical check-up is to understand an individual's health conditions and detect early symptoms or risk factors, and have treatments at an early stage. However, a general medical check-up is just applicable for examining whether or not there is a change in an organ, but it cannot prevent the change of the organ in advance, so that people usually see a doctor or seek for medical help and treatment when they are already ill and the patients may fail to recover their original health status or even have sequelae or disabilities. Therefore, preventive medicine becomes increasingly important and focuses on preserving, promoting, and maintaining the health of the general public, a specific group, or an individual. The preventive medicine primarily includes health promotion and disease prevention, wherein the health promotion mainly covers lifestyle, living environment, and healthcare system, and disease prevention mainly covers the areas of assessing and improving a person's health based on the risk factors of health before one gets sick, and further restrict the development of the disease.

In view of the aforementioned problems, the inventor of the present invention conducted extensive researches and experiments, and finally provided a feasible solution to overcome the problems of the prior art.

### SUMMARY OF THE INVENTION

Therefore, it is a primary objective of the present invention to provide a human body frequency diagnostic analysis apparatus and an improvement method of health assessment capable of diagnosing and analyzing human body frequencies and further examining a person's health condition.

To achieve the aforementioned objective, the present invention provides a human body frequency diagnostic analysis apparatus comprising a transceiver, a detection unit and a control unit, wherein the transceiver has an end coupled to a human head and capable of being driven to transmit a transmission frequency and drive the human head to feed back a feedback frequency from the transceiver; the detection unit is coupled to the transceiver for driving the transceiver to transmit the transmission frequency and receive the feedback frequency; the control unit is coupled to the detection unit for controlling the detection unit; the control unit further comprises a computing unit, a database and a screen, wherein the computing unit is provided for computing and comparing the transmission frequency with the feedback frequency to generate a dual line spectrum, and a maximum drop value in a frequency section of the dual line spectrum is detected, and the maximum drop value indicates a human body frequency imbalance; the database is provided for comparing the dual line spectrum to generate detected information; and the screen is provided for displaying the dual line spectrum and the detected information.

The human body frequency diagnostic analysis apparatus further comprises a heartbeat sensor coupled to a human pulse (or a heart position) to obtain a number of heartbeats, and the control unit has an input unit for inputting the number of heartbeats, and generating a plurality of predicted frequency values after the computing unit computes the inputted number of heartbeats, and further comparing the predicted frequency value with a maximum drop value in a frequency section of the dual line spectrum, and the maximum drop value indicates a human body meridian frequency imbalance.

In the human body frequency diagnostic analysis apparatus, the human body frequency diagnostic analysis apparatus is capable of detecting a frequency section from 1.8 Hz to 8.2 Hz.

Another objective of the present invention is to provide an improvement method of human health assessment, comprising the steps of: connecting an end of a transceiver to a human head, and driving the transceiver to transmit a transmission frequency, and further driving the human head to feed back a feedback frequency through the transceiver; using a detection unit and the transceiver to drive the transceiver to transmit the transmission frequency and receive the feedback frequency; connecting a control unit to the detection unit to control the detection unit, wherein the control unit further comprises a computing unit, for computing the transmission frequency and the feedback frequency, and generates a dual line spectrum, and a maximum drop value occurred in a frequency section of the dual line spectrum indicates an imbalance of a human body frequency, and the control unit includes a database used for comparing the dual line spectrum and generating detected information, and the control unit further includes a screen for displaying the dual line spectrum and the detected information.

In the improvement method of human health assessment, the improvement method of human health assessment is capable of detecting a frequency from 1.8 Hz to 8.2 Hz.

The improvement method of human health assessment further comprises a step of connecting a heartbeat sensor to a human pulse (or a heart position) to obtain a number of heartbeats; wherein the control unit has an input unit for inputting the number of heartbeats, and generating a plurality of predicted frequency values after the computing unit computes the inputted number of heartbeats, and further compares the predicted frequency value with a maximum drop value in a frequency section of the dual line spectrum, and the maximum drop value indicates a human body meridian frequency imbalance.

The improvement method of human health assessment may be used together with a rhythm machine capable of generating a rhythm wave, and when the rhythm machine is contacted with a human body, the rhythm machine generates a rhythm wave with the maximum drop value to achieve the purpose of improving the human body frequency.

The improvement method of human health assessment may be applied together with an essential oil to achieve the effect of improving human health.

### BRIEF DESCRIPTION OF THE DRAWINGS

FIG. 1 is a block diagram of a preferred embodiment of the present invention;

FIG. 2 is a flow chart of detecting human frequencies in accordance with a preferred embodiment of the present invention;

FIG. 3 is a flow chart of detecting a human body meridian frequency in accordance with a preferred embodiment of the present invention;

FIG. 4 is a reference diagram of a dual line spectrum of a preferred embodiment of the present invention; and

FIG. 5 is a reference diagram of a human body meridian frequency of a preferred embodiment of the present invention.

### DETAILED DESCRIPTION OF THE PREFERRED EMBODIMENTS

The technical characteristics, contents, advantages and effects of the present invention will be apparent with the detailed description of a preferred embodiment accompanied with related drawings as follows.

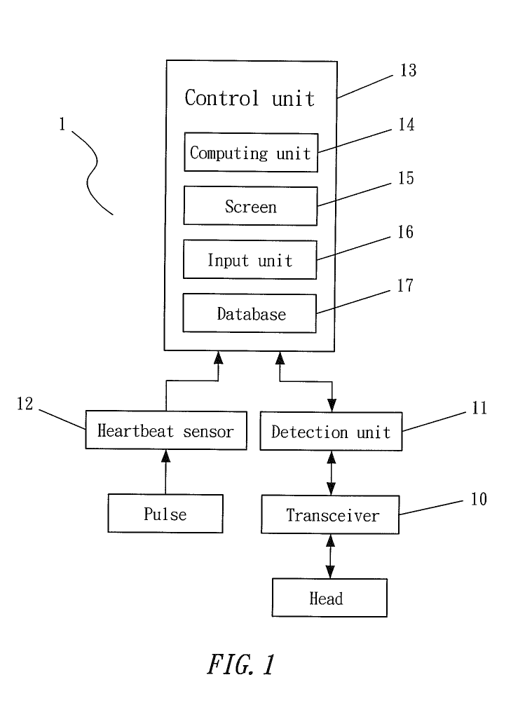

With reference to FIG. 1 for a human body frequency diagnostic analysis apparatus 1 of the present invention, the human body frequency diagnostic analysis apparatus 1 comprises:

a transceiver 10, with an end coupled to a human head, and capable of being driven to transmit a transmission frequency, and drive the human head to feed back a feedback frequency from the transceiver 10;

a detection unit 11, coupled to the transceiver 10, for driving the transceiver 10 to transmit the transmission frequency and receive the feedback frequency;

a heartbeat sensor 12, coupled to a human pulse (or a heart position) to obtain a number of heartbeats; and

a control unit 13, coupled to the detection unit 11, for controlling the detection unit 11;

wherein, the control unit 13 comprises a computing unit 14, a screen 15, an input unit 16 and a database 17, and the computing unit 14 is provided for computing the transmission frequency and the feedback frequency to generate a dual line spectrum; the input unit 16 is provided for inputting the number of heartbeats, and using the computing unit 14 to compute the inputted number of heartbeats to generate a plurality of predicted frequency values; the database 17 is provided for comparing the dual line spectrum to generate detected information; and the screen 15 is provided for displaying the dual line spectrum and the detected information.

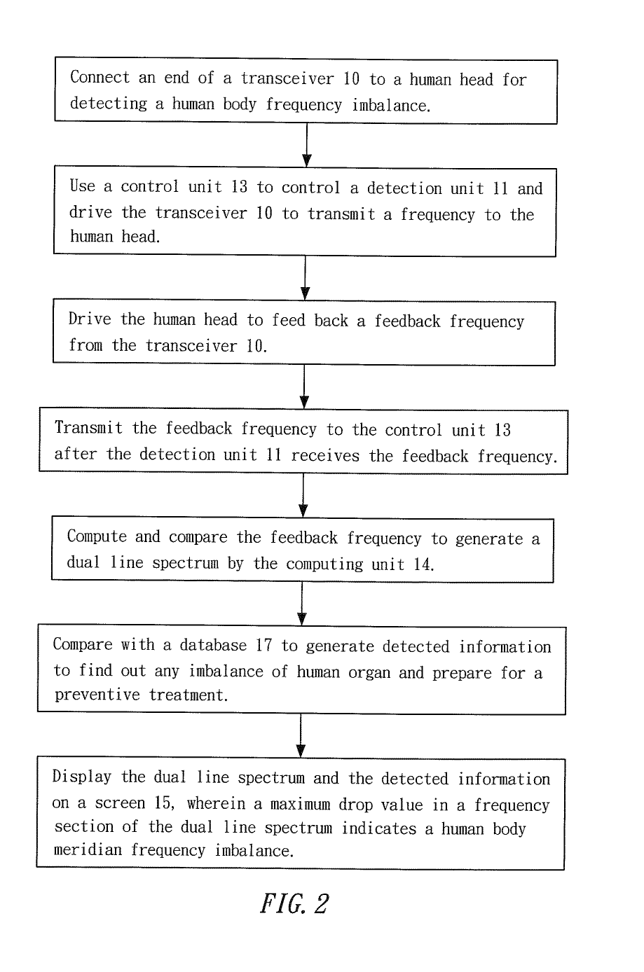

With reference to FIGS. 1 and 2 for the detection of a human body frequency imbalance, an end of the transceiver 10 is connected to a human head, and the control unit 13 is provided for controlling the detection unit 11 and driving the transceiver 10 to transmit the transmission frequency to the human head, and further driving the human head to feed back the feedback frequency from the transceiver 10. After the detection unit 11 receives the feedback frequency, the feedback frequency is transmitted to the control unit 13, and the computing unit 14 computes and compares the transmission frequency with the feedback frequency to generate the dual line spectrum, and further compares with the database 17 to generate the detected information to discover any human organ imbalance and prepare a preventive treatment, and the dual line spectrum and the detected information are displayed on the screen 15. It is noteworthy that the maximum drop value in the frequency section of the dual line spectrum indicates a human body frequency imbalance (as shown in FIG. 4, the maximum drop value is located at the position of 6.6 Hz).

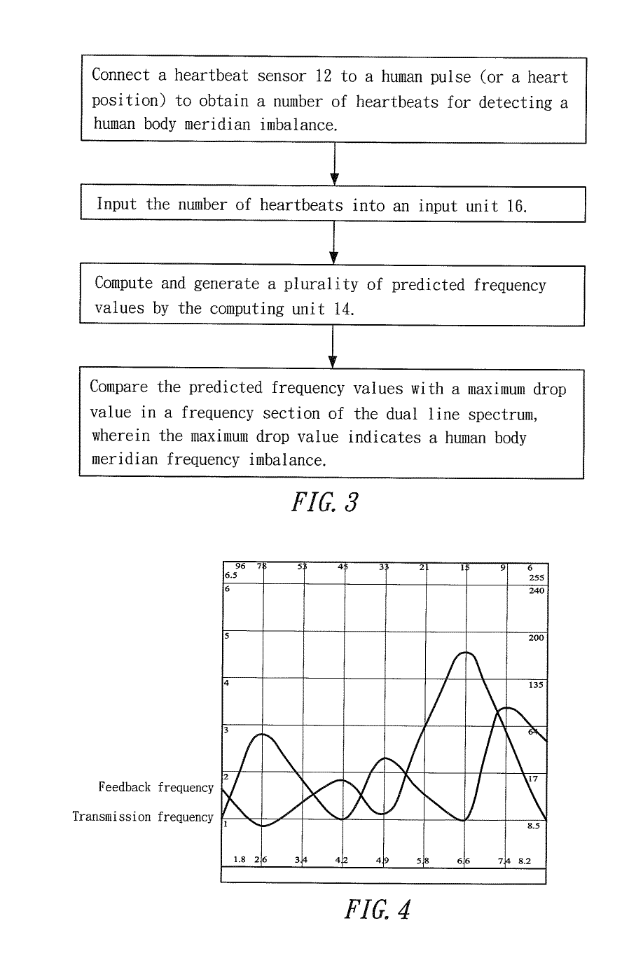

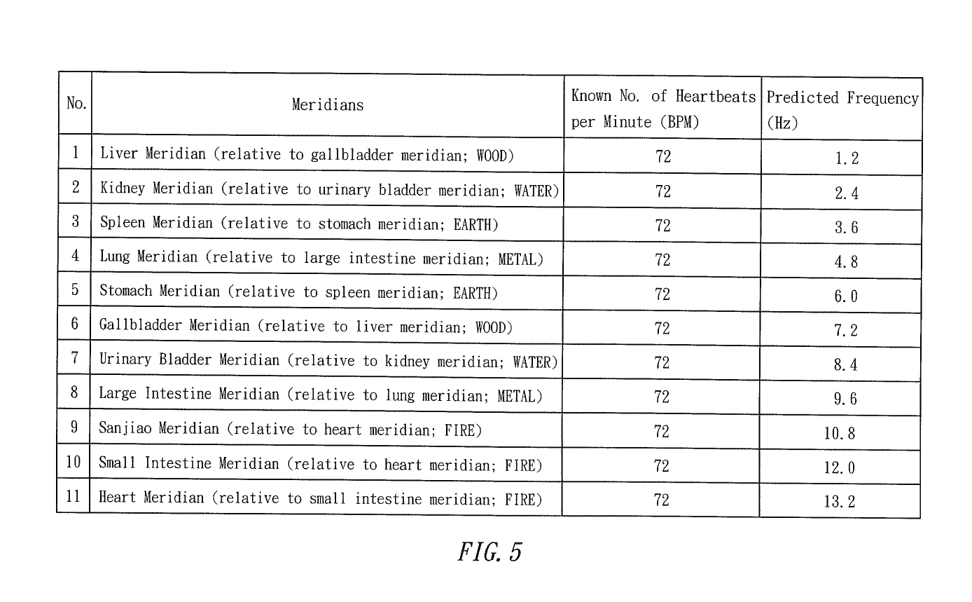

With reference to FIGS. 1 and 3 for the detection of a human body meridian frequency imbalance, the heartbeat sensor 12 is connected to a human pulse (or a heart position) to obtain the number of heartbeats, and the number of heartbeats is inputted into the input unit 16. After the computing unit 14 computes to generate a plurality of predicted frequency values, the predicted frequency value is compared with the maximum drop value in the frequency section of the dual line spectrum, wherein the maximum drop value indicates a human body meridian frequency imbalance. In FIG. 5, the number of heartbeats of human body equal to 72 is inputted, and a plurality of predicted frequencies is obtained after computation, and the predicted frequency is compared with the dual line spectrum. The maximum drop value is situated at a position of 6.6 Hz, and thus showing that the human stomach meridians have problems.

The improvement method of human health assessment of the present invention may be applied in the human body frequency diagnostic analysis apparatus, and the improvement method of human health assessment comprises the following steps:

Connect an end of a transceiver to a human head, and drive the transceiver to transmit a transmission frequency and further drive the human head to feed back a feedback frequency from the transceiver.

Use a detection unit and the transceiver to drive the transceiver to transmit the transmission frequency and receive the feedback frequency.

Connect a heartbeat sensor to a human pulse (or a heart position) to obtain a number of heartbeats.

Connect a control unit to the detection unit to control the detection unit, wherein the control unit, wherein the control unit further comprises a computing unit, for computing the transmission frequency and the feedback frequency, and generates a dual line spectrum, and a maximum drop value occurred in a frequency section of the dual line spectrum indicates an imbalance of a human body frequency, and the control unit includes a database used for comparing the dual line spectrum and generating detected information, and the control unit further includes a screen for displaying the dual line spectrum and the detected information. The control unit has an input unit for inputting the number of heartbeats, and generating a plurality of predicted frequency values after the computing unit computes the inputted number of heartbeats, and further compares the predicted frequency value with a maximum drop value in a frequency section of the dual line spectrum, and the maximum drop value indicates a human body meridian frequency imbalance. Therefore, the human meridian is imbalanced and a preventive treatment is required.

In addition, a rhythm machine capable of generating a rhythm wave may be used and contacted with a human body, and the rhythm machine can adjust the frequency of the rhythm wave. For example, when the maximum drop value is situated at the position of 6.6 Hz, the frequency of the rhythm machine is adjusted to 6.6 Hz, so that the rhythm machine will output a rhythm wave of 6.6 Hz to adjust the human body frequency.

Further, an essential oil may be used in the method to improve the imbalance of human organs or meridians.

Therefore, an imbalance of human organ or meridian frequency and the health condition of a patient can be detected, and the human body or meridian frequency can be adjusted.

In summation of the description above, the present invention achieves the expected functions and objectives, and complies with the patent application requirements, and thus is duly filed for patent application. While the invention has been described by means of specific embodiments, numerous modifications and variations could be made thereto by those skilled in the art without departing from the scope and spirit of the invention set forth in the claims.

What is claimed is:

1. A human body frequency diagnostic analysis apparatus, comprising:
a transceiver, with an end coupled to a human head, and capable of being driven to transmit a transmission frequency, and drive the human head to feed back a feedback frequency from the transceiver;
a detection unit, coupled to the transceiver, for driving the transceiver to transmit the transmission frequency and receive the feedback frequency; and
a control unit, coupled to the detection unit, for controlling the detection unit, and the control unit further comprising a computing unit, for computing the transmission frequency and the feedback frequency, and generating a dual line spectrum, wherein a maximum drop value occurred in a frequency section of the dual line spectrum indicates an imbalance of a human body frequency, and the control unit includes a database used for comparing the dual line spectrum and generating detected information, and the control unit further includes a screen for displaying the dual line spectrum and the detected information, and an imbalance of a human organ or a human meridian may be detected for a preventive treatment.

2. The human body frequency diagnostic analysis apparatus according to claim 1, further comprising a heartbeat sensor, coupled to a human pulse (or a heart position) to obtain a number of heartbeats; the control unit having an input unit for inputting the number of heartbeats, and generating a plurality of predicted frequency values after the computing unit computes the inputted number of heartbeats, and further comparing the predicted frequency value with a maximum drop value in a frequency section of the dual line spectrum, and the maximum drop value indicating a human body meridian frequency imbalance.

3. The human body frequency diagnostic analysis apparatus according to claim 1, wherein the human body frequency diagnostic analysis apparatus is capable of detecting a frequency section from 1.8 Hz to 8.2 Hz.

4. The human body frequency diagnostic analysis apparatus according to claim 1, wherein the human body frequency diagnostic analysis apparatus is operated together with a rhythm machine capable of generating a rhythm wave, and when the rhythm machine is contacted with a human body, the rhythm machine generates a rhythm wave with the maximum drop value to achieve the purpose of improving the human body frequency.

5. The human body frequency diagnostic analysis apparatus according to claim 1, wherein the human body frequency diagnostic analysis apparatus is used together with an essential oil to achieve the effect of improving human health.

6. An improvement method of human health assessment, comprising the steps of:
connecting an end of a transceiver to a human head, and driving the transceiver to transmit a transmission frequency, and further driving the human head to feed back a feedback frequency through the transceiver;
using a detection unit and the transceiver to drive the transceiver to transmit the transmission frequency and receive the feedback frequency;
connecting a control unit to the detection unit to control the detection unit, wherein the control unit further comprises a computing unit, for computing the transmission frequency and the feedback frequency, and generates a dual line spectrum, and a maximum drop value occurred in a frequency section of the dual line spectrum indicates an imbalance of a human body frequency, and the control unit includes a database used for comparing the dual line spectrum and generating detected information, and the control unit further includes a screen for displaying the dual line spectrum and the detected information.

7. The improvement method of human health assessment according to claim 6, further comprising the step of connecting a heartbeat sensor to a human pulse (or a heart position) to obtain a number of heartbeats; wherein the control unit has an input unit for inputting the number of heartbeats, and generating a plurality of predicted frequency values after the computing unit computes the inputted number of heartbeats, and further compares the predicted frequency value with a maximum drop value in a frequency section of the dual line spectrum, and the maximum drop value indicates a human body meridian frequency imbalance.

8. The improvement method of human health assessment according to claim 6, wherein the improvement method of human health assessment is capable of detecting a frequency from 1.8 Hz to 8.2 Hz.

9. The improvement method of human health assessment according to claim 6, wherein the improvement method of human health assessment is used together with a rhythm machine capable of generating a rhythm wave, and when the rhythm machine is contacted with a human body, the rhythm machine generates a rhythm wave with the maximum drop value to achieve the purpose of improving the human body frequency.

10. The improvement method of human health assessment according to claim 6, wherein the improvement method of human health assessment is applied together with an essential oil to achieve the effect of improving human health.
